(19) United States
(12) Patent Application Publication
(10) Pub. No.: US 2016/0271416 A1
(43) Pub. Date: Sep. 22, 2016

(54) SLEEP MASK THAT INCORPORATES LIGHT TO REGULATE UTERINE CONTRACTIONS

(71) Applicant: FLORIDA STATE UNIVERSITY RESEARCH FOUNDATION, INC., Tallahassee, FL (US)

(72) Inventor: James OLCESE, Tallahassee, FL (US)

(21) Appl. No.: 15/024,508

(22) PCT Filed: Nov. 4, 2014
(86) PCT No.: PCT/US14/63877

§ 371 Date: Mar. 24, 2016

Related U.S. Application Data
(60) Provisional application No. 61/903,497, filed on Nov. 13, 2013.

(51) Int. Cl. A61N 5/06

(52) U.S. Cl. CPC A61N 5/0613; A61N 2005/0626; A61N 2005/0648; A61N 2005/0651; A61N 2005/0663

(57) ABSTRACT

A sleep mask includes a flexible eye cover configured to be worn by a pregnant female and configured to cover and shield the eyes of the wearer from ambient light. A source of light is carried by the flexible eye cover and positioned to emit light onto the eyelids of the wearer when asleep and penetrate the eyelids when closed. A processor is connected to the source of light and configured to activate the light in a manner to decrease endogenous melatonin levels and aid in regulating uterine contractions in the pregnant female.

### CROSS-REFERENCE TO RELATED APPLICATIONS)

This is a PCT application based on provisional application Ser. No. 61/903,497 filed Nov. 13, 2013, the disclosure which is hereby incorporated by reference in its entirety.

### FIELD OF THE INVENTION

The invention relates to the field of pregnancy and, more particularly, to methods of regulating uterine contractions.

### BACKGROUND OF THE INVENTION

In Western societies preterm labor occurs in more than 12% of all pregnancies. It remains a major cause of perinatal morbidity and is associated with 70% of neonatal mortality. According to the Institute of Medicine of the National Academies of Sciences, the economic burden of preterm births in the United States is well over $26 billion per year (>$100,000 per infant). Despite this continually increasing medical challenge there has been relatively little progress in the past 20 years in understanding the processes initiating labor, whether term and preterm.

There is no test to accurately predict preterm labor. The primary goal in preventing preterm birth is to eliminate the high risk of neonatal mortality and neonatal complications (especially in terms of pulmonary and brain function). Among the major pharmacological approaches for treating preterm labor are oxytocin receptor antagonists (Atosiban®; Tractocile®), β-adrenergic receptor agonists, cyclooxygenase (COX) inhibitors, nitric oxide donors and magnesium sulfate. Although some of these agents have modest success as tocolytics, all have considerable, sometimes serious, side effects which can limit their use. Although progesterone has been used to prevent preterm labor in women at risk, most preterm births occur in women with no significant risk factors.

Clearly, this socio-medically important problem is far from resolved. Understanding the molecular mechanisms of labor thus should have a high priority in biomedicine. There is evidence of a synergistic action between melatonin receptor activation and oxytocin-induced signaling that may provide a key hormonal event in the initiation of labor. Ramifications of these findings for the practice of obstetrics could be dramatic. For example, the blockade of melatonin receptor activity might be of great value in preventing preterm labor and thus extending pregnancy to improve the chances of optimal survival for the newborn.

U.S. Pat. No. 8,445,436 by the same inventor, the disclosure which is hereby incorporated by reference in its entirety, discloses that the brain hormones melatonin and oxytocin use similar intracellular mechanisms in promoting contraction of human myometrium smooth muscle cells. Oxytocin analogues are important tools in obstetrical practice, e.g. infusion of oxytocin analogues is commonly used to induce labor, while oxytocin antagonists are used to prolong pregnancy in cases of preterm labor (although they are only minimally effective). A significant positive synergistic action of melatonin and oxytocin exists on human myometrial cell contractions in vitro, such that in the presence of melatonin even as little as 1% of the oxytocin dose normally needed for maximal contraction is fully effective. These findings could lead to the development of new melatonin plus low dose oxytocin combinations for labor induction without the mentioned side effects of high oxytocin levels.

Signaling through uterine melatonin receptors may actively contribute to labor by serving to temporally “gate” the events leading to uterine contractions at night. Endogenous melatonin may normally act synergistically with oxytocin (and potentially other pro-contractile factors) to facilitate the coordinated and forceful contractions of the pregnant uterus necessary for term labor. By extension, expression of these receptors prematurely in the myometrium of pregnant women may contribute to preterm labor.

To show that removing melatonin's drive to the pregnant myometrium can lower uterine contractions during pregnancy, clinical studies had been completed in which >39 week term pregnant volunteers were recruited. The volunteers were continuously monitored for uterine contractions from 7:00 p.m. until 7:00 a.m. under dim light. At 11:00 p.m., a 10,000 lux full spectrum lamp 1 meter from the head was activated for 1 hour to suppress melatonin secretion. This study is disclosed in U.S. patent application Ser. No. 13/938,485 and published as U.S. Patent Publication No. 2014/0094877, the disclosure which is hereby incorporated by reference in its entirety.

As shown in FIGS. 1-3 (representative of 18 volunteers assessed), nocturnal contraction frequency rises early in the night, only to be suppressed by bright light exposure. Once endogenous melatonin levels return to high nocturnal levels (2-3 hours later), uterine contractions are again near maximal. These results support the view that melatonin plays a key role in the uterine contractions of labor. It would be advantageous if a light emitting device could be used to treat preterm labor and is amenable for use by a pregnant woman at night during sleep.

### SUMMARY OF THE INVENTION

This summary is provided to introduce a selection of concepts that are further described below in the detailed description. This summary is not intended to identify key or essential features of the claimed subject matter, nor is it intended to be used as an aid in limiting the scope of the claimed subject matter.

A sleep mask includes a flexible eye cover configured to be worn by a pregnant female and configured to cover and shield the eyes of the wearer from ambient light. A source of light is carried by the flexible eye cover and positioned to emit light onto the eyelids of the wearer when asleep and penetrate their eyelids when closed. A processor is connected to the source of light and configured to activate the light in a manner to decrease endogenous melatonin levels and aid in regulating uterine contractions in the pregnant female.

In one example, the wavelength of the source of light is between about 450 to 500 nm. The light irradiance is between about 80 to 140 W/m². In another example, a battery is carried by the flexible eye cover and connected to and supplies power to the processor and source of light. In another example, a wireless power source is included and the processor and source of light receives wireless power transmitted from the wireless power source. It is possible that only the source of light as LED's receives the wireless power. The illumination of the light is increased over a predetermined period of time such as between about 10 to 30 seconds. In another example, the light is pulsed in discrete on and off cycles such as between about 1 to 3 seconds per minute for about 30 to 60 minutes. The flexible eye cover in an example may be formed from fabric. The source of light may be formed as a light emitting diode (LED) carried by the flexible eye cover and positioned at a location where a LED can emit light onto the eyelids of the wearer when asleep.

In another example, the flexible eye cover is configured to be worn by a pregnant female and has a rear surface that engages the face of the wearer and configured to cover and shield the eyes of the wearer from ambient light when worn by a wearer. A front surface is opposite the rear surface. A pocket is formed on the rear surface at each location in an area generally where the eye cover engages the eyes. A light emitting diode (LED) emits light having a wavelength between about 450 to 500 nm and is carried by the flexible eye cover within each pocket and moveable within the pocket to be positioned to emit light onto the eyelids of the wearer when asleep. A processor is connected to each LED and configured to activate the LED in a manner to decrease endogenous melatonin levels and aid in regulating uterine contractions in a pregnant female. In an example, the LED is adjustable by the user to position the LED near an eyelid. For example, an adjuster member is connected to each LED and extends outward from the front face of the flexible eye cover and is configured to be engaged manually by the wearer to manipulate and move a respective LED into a position adjacent an eye to ensure that the light from the LED is emitted onto the closed eyelid of the wearer when asleep.

A method of regulating uterine contractions in a pregnant female includes providing a flexible eye cover that is worn by the pregnant female and covers and shields the eyes of the pregnant female from ambient light. The method includes emitting light from a light source carried by the flexible eye cover onto the eyelids of the pregnant female when asleep to penetrate the eyelids when closed. The method further includes activating the light in a manner to decrease endogenous melatonin levels to aid in regulating uterine contractions in the pregnant female.

### BRIEF DESCRIPTION OF THE DRAWINGS

FIG. 1 is a bar graph showing the number of contractions per hour a pregnant volunteer experienced overnight and how the number changed when a lamp was turned on for about one hour.

FIG. 2 is a bar graph showing the number of contractions per hour that another pregnant volunteer experienced overnight and how the number changed when a lamp was turned on for about one hour.

FIG. 3 is a line graph showing composite results of the melatonin levels of five pregnant volunteers when a lamp was turned on for about an hour.

FIG. 4 is a fragmentary perspective view of a user wearing a pair of goggles/glasses that may be worn by a pregnant female during nocturnal hours to regulate uterine contractions while she sleeps.

FIG. 5 is a rear fragmentary view of a sleep mask formed as a flexible eye cover that can be worn by a pregnant female and includes a source of light as LED's and a processor that activates the light in a manner to decrease endogenous melatonin levels to aid in regulating uterine contractions in a pregnant female.

FIG. 6 is a front fragmentary view of the sleep mask showing part of the fabric forming the flexible eye cover and broken away to show the position of a processor and battery and relative positioning of the LED's and also showing a separate wireless power source and controller that may be used in conjunction with the sleep mask.

### DETAILED DESCRIPTION

In the Summary above and in the Detailed Description, reference is made to particular features (including method steps) of the invention. Where a particular feature is disclosed in the context of a particular aspect or embodiment of the invention, that feature can also be used, to the extent possible, in combination with and/or in the context of other particular aspects and embodiments of the invention, and in the invention generally.

As discussed above, the various method aspects of the invention are directed to regulating uterine contractions by exposing pregnant females to light. The term “regulating,” in this context means reducing the number of uterine contractions over a given time period, reducing the intensity of the uterine contractions, and or preventing uterine contractions from occurring when they might otherwise occur in the absence of light. These methods are useful, for example, to prolong pregnancy, prevent preterm birth, or, if preterm birth is inevitable, to delay the preterm birth.

As noted in the incorporated by reference '485 patent application serial number corresponding to the '877 patent publication, parturition is a physiological process that occurs when pregnant females are in labor. It is characterized by increasingly frequent uterine contractions and cervical effacement, which ultimately leads to delivery of offspring. Parturition is a complex physiological and molecular biological process that has evolved differently in different species due to each species' unique environmental and temporal niches. Most mammals have adapted to selective pressures, such as the availability of food and prevalence of predators, by developing either a diurnal or nocturnal activity phase. Pregnant females have adapted to deliver their offspring in their den or home camp rather than in the field, which enhances their safety, security, and birth success.

The selective advantage for entering parturition during the daytime or nighttime phases is reflected in the differential timing of this event among many species of nocturnal rodents and diurnal mammals, such as sheep and primates. Rats, for example, enter parturition predominantly during the day time, even when the light-dark cycles are reversed.[1-3] Similarly, golden hamsters develop strong uterine contractions and deliver their young during daytime hours.[4] Humans, on the other hand, tend to enter labor during the late nighttime and early morning hours [5-9] with parturition typically following 12 to 24 hours thereafter, at least in nulliparous women.[7] The frequency of uterine contractions in preterm women at risk for spontaneous premature delivery increases significantly at night.[10]

In non-human primates, the late-term myometrial contractions and the sensitivity of the uterus to the contractile effects of oxytocin have been shown to be the highest early in the night phase.[11-13] In addition, the phasing of nocturnal parturition in nonhuman primates has been shown to also be shifted by reversal of the light/dark cycles [14], pointing to a light-sensitive clock mechanism underlying parturition. Since both humans and nonhuman primates show nocturnally peaking uterine contractions in late-term pregnancy [15-17], the intriguing question arises—what are the circadian signals that drive nocturnal uterine activity in late term human pregnancy?

Maintenance of appropriate circadian phase in peripheral tissues requires zeitgebers (entraining cues) that are coupled with the central circadian oscillator in the brain's suprachiasmatic nuclei (SCN) via neural pathways, rhythmic endocrine, and/or metabolic signals. For many peripheral clocks, such as the liver, heart, pancreas, and so on, autonomically driven neuroendocrine output cues such as melatonin and glucocorticoids are considered to play a key role. [18] Evidence continues to accumulate showing that these two hormones have significant effects on the endogenous circadian clockwork in a variety of peripheral tissues.[19-24]

It is known that the endogenous melatonin level of a typical person rises gradually from about 9:00 p.m. to a maximum at about 2:00 am. After about 2:00 a.m., the endogenous melatonin level gradually decreases until morning and remains very low throughout the day. The cycle then repeats itself the following night. Because the endogenous melatonin level reaches its peak at night, this is the time period during which melatonin stimulates the most contractions. By exposing a pregnant female to a light source with sufficient intensity to suppress the endogenous melatonin level, uterine contractions are suppressed. In this context “suppressing” the endogenous melatonin level refers to either reducing the endogenous melatonin level from normal or preventing the endogenous melatonin level from rising as it normally would at night.

As described in the incorporated by reference '485 patent application serial number corresponding to the '877 publication, clinical experiments were performed on actual pregnant female human patients. Pregnant female human volunteers at >38 weeks of gestation were studied to determine whether exposing them to visible light would suppress their uterine contractions during the nighttime hours and whether the suppression of uterine contractions that might result from light exposure is correlated with a decrease in their endogenous melatonin levels. In the experiments, women were studied by continuously monitoring their uterine contractions from 7:00 p.m. until 7:00 am under dim light. At 11:00 p.m., each woman was exposed to a 10,000 lux, full spectrum phototherapy lamp positioned about 1 meter from the woman's eyes. After about 1 hour, the lamp was turned off. The contractions were recorded by a registered obstetric nurse. The study was performed in a hospital after receiving approval from the appropriate institutional review boards.

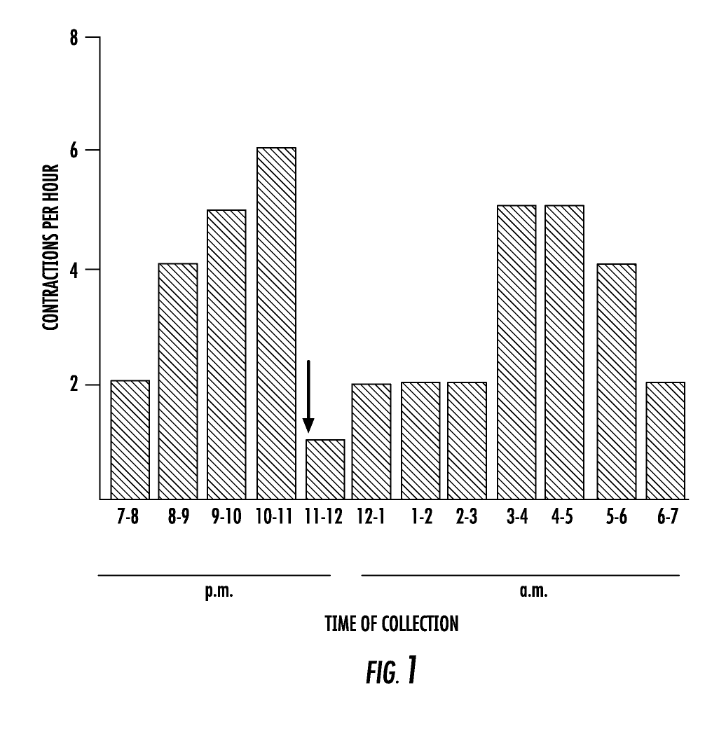

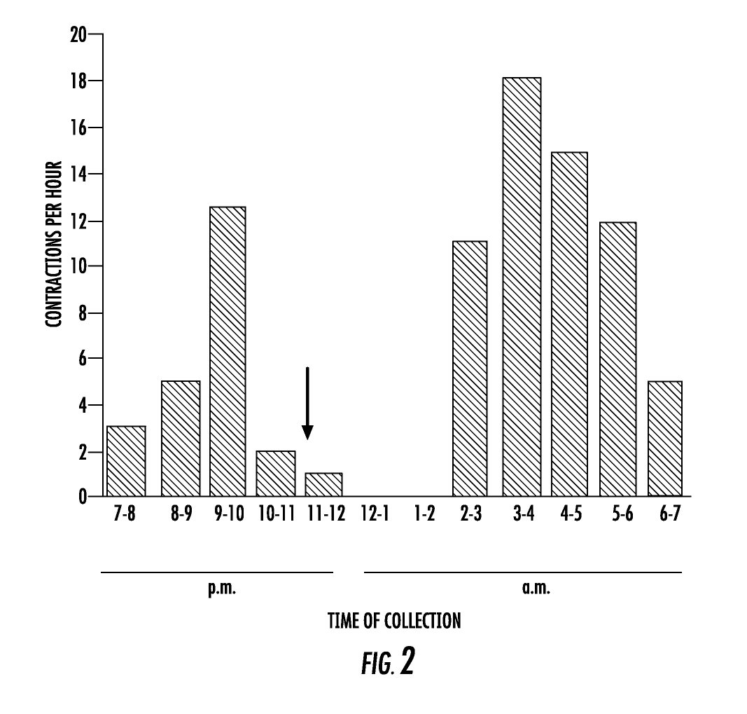

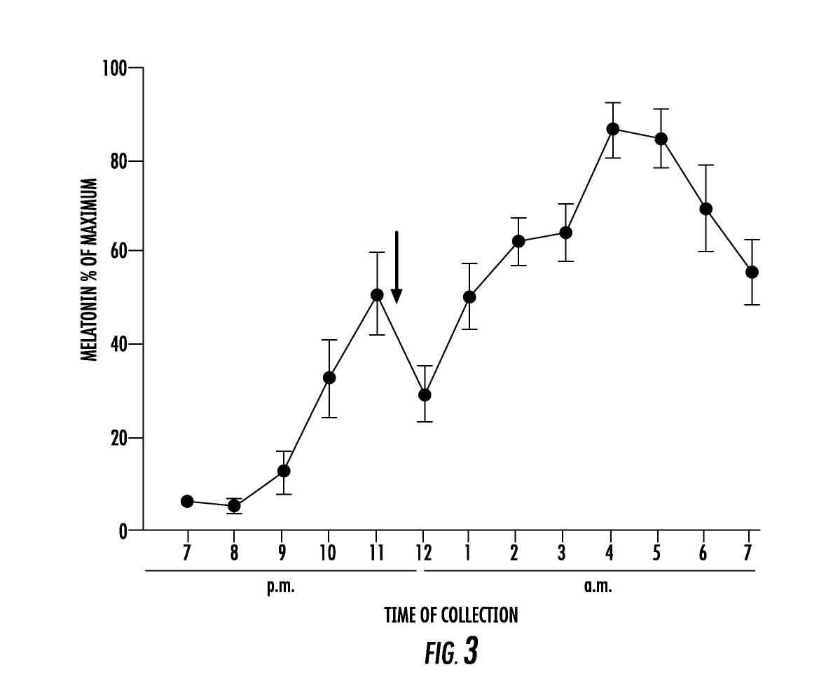

The results of these experiments are shown in FIGS. 1-3. The number of contractions two different women experienced per hour are shown in FIGS. 1 and 2. FIG. 3 shows the composite results of the melatonin levels of the women studied. The arrow in each graph represents the time at which the lamp was turned on.

The results show that, when the lamp was turned on at about 11:00 p.m., the number of contractions experienced by the women per hour decreased substantially. When the lamp was turned off after about 1 hour, the number of contractions the women experienced per hour gradually rose before eventually decreasing during the early morning hours. FIG. 3 shows that the women's endogenous melatonin levels gradually rose until 11:00 p.m. when the lamp was turned on and dropped by about 45% during the ˜1 hr that the lamp was activated. Once the lamp was turned off, their melatonin levels gradually rose throughout the night before decreasing during the early morning hours. When combined, the results indicate that there is indeed a direct correlation between melatonin levels and uterine contractions.

The results reveal that regular nocturnal contractions are suppressed by bright light exposure under these conditions. This finding supports the proposition that melatonin is a key zeitgeber, regulating the onset of human labor and parturition and that light can be used to regulate melatonin levels and, thereby, regulate uterine contractions.

Thus, a method of regulating uterine contractions involves suppressing the nocturnal endogenous melatonin level of a pregnant female experiencing uterine contractions by exposing the pregnant female during nighttime to a light source emitting visible light such as using glasses, sleep mask or other device. The intensity of the visible light is sufficient to suppress a pregnant female's endogenous melatonin level. It is also possible to regulate nocturnal uterine contractions during preterm labor by exposing a pregnant female experiencing preterm labor to a light source at night, where the light source emits visible light effective to suppress the pregnant female's endogenous melatonin level.

The light source should be of sufficient intensity and color to be able to suppress the endogenous melatonin level. In the experiment as described, the light source was a full spectrum 10,000 lux phototherapy lamp positioned about 1 meter from the pregnant female's eyes. Although this yielded good results, other light sources are suitable for use. A suitable intensity range for the light source is about 1,000 to about 10,000 lux.

The light source spectrum may be tuned to optimize the amount of melatonin suppression. One preferred light source predominantly emits blue light. Blue light in the wavelength range of about 450 to about 500 nm has been found advantageous. In one example, the pregnant female is exposed to the light during typical nocturnal or nighttime hours, preferably between about 9 p.m. to about 6 a.m. The pregnant female may be exposed to the light source continuously throughout the night or in smaller time increments during the night.

Optionally, the light source is adapted to emit light in discrete on/off cycles or pulses. The duration of the pulses and the separation between successive pulses is adjusted to obtain the desired amount of endogenous melatonin suppression.

“A Train of Blue Light Pulses Delivered Through Closed Eyelids Suppresses Melatonin and Phase Shifts the Human Circadian System,” by Figueiro et al., Nature and Science of Sleep, 2013:5, 133-141, the disclosure which is hereby incorporated by reference in its entirety, teaches that a train of blue light pulses delivered through closed eyelids suppresses melatonin and phase shifts the human circadian system. [25]

It is possible to use a personal light-emitting device for women at risk of preterm labor. Such light-emitting devices such as goggles or other glasses have already been constructed and tested with non-pregnant volunteers for sleep studies such as at Rensselaer Polytechnic Institute in Troy, N.Y. The light mask included goggles with machined aluminum heat sinks to dissipate heat generated by light sources. The light generated by the mask was controlled by a program that increased light levels gradually over two minutes from zero to a prescribed light level with the assumption that the temporal ramp would avoid “startling” the subjects with the light dose while they were asleep. This study describes in the open access short report by Mariana G. Figueiro and Mark S. Rea entitled, “Preliminary Evidence that Light Through the Eyelids can Suppress Melatonin and Phase Shift Dim Light Melatonin Onset,” BMC Research Notes, 2012, 5:221, http://www.biomedicalcentral.com/1756-0500/5/221, the disclosure which is hereby incorporated by reference in its entirety.

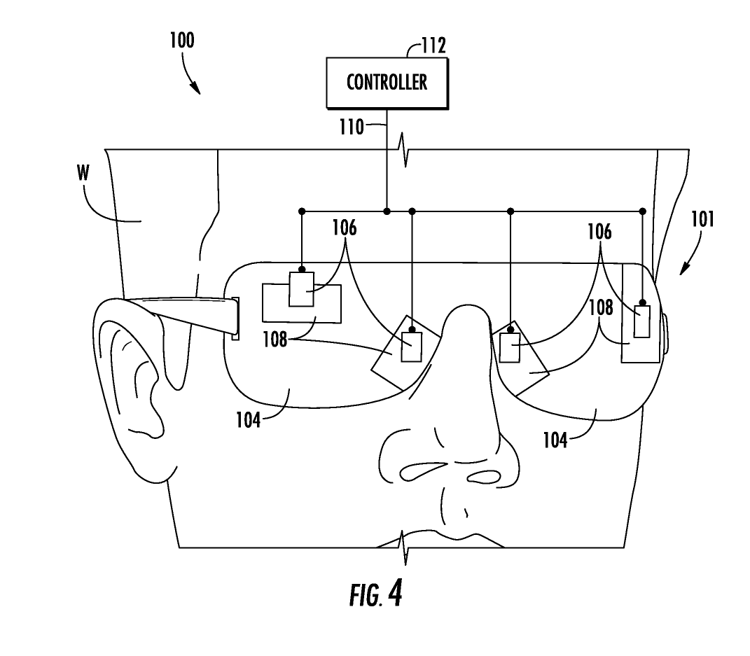

FIG. 4 shows an example of a preferred light source 100 used with a subject such as a pregnant female. The light source is formed a glasses 101 in this example, including a frame 102 supporting a pair of transparent lenses 104. Two lights 106 are attached to each lens 104 and are oriented to shine their light towards a wearer's W eyes. Light diffusers 108 on the lenses 104 diffuse the light so that the lights do not appear as intense point sources of light to the wearer W. The diffusers 108 are made of translucent material. In this example, standard glasses may be modified.

Electrical wiring 110 in one example connects the LEDs 106 to a controller 112 that powers and controls the lights 106. The controller 112 turns the lights 106 on and off, controls the intensity of the emitted light, and, if desired turns the lights 106 on and off in discrete on/off cycles or pulses. The controller 112 is also configured to gradually provide power to the lights 106 so that the lights 106 gradually illuminate over time. This feature is designed to prevent the wearer from waking up when the lights 106 are turned on. The lights 106 are preferably LEDs.

The controller in one example includes a source of power such as a battery. The controller can be mounted directly on the frame 102 in a position where it will not bother the wearer such as extending outward from the side of the frame between the ear and lens. In another example, the controller could be powered from a wireless power source. In yet another example, a wireless power source could be used and energy supplied directly to the lights, which could be formed as LED's that include a wireless power receiver, as an example.

The light source 100 may be worn by a pregnant female during nocturnal hours to regulate her uterine contractions, even while she sleeps. In an embodiment, the light is pulsed in discrete on/off cycles. A pulse cycle is a pulse of light for between 1-3 seconds per minute for at least about 60 minutes. Another pulse cycle is a pulse of light for about 2 seconds per minute for at least about 60 minutes. An example wavelength is between 450-500 nm, in an example, about 470 nm to about 490 nm, or about 480 nm. The light irradiance is preferably between about 80-140 W/m², and between about 105-115 W/m², or about 111 W/m².

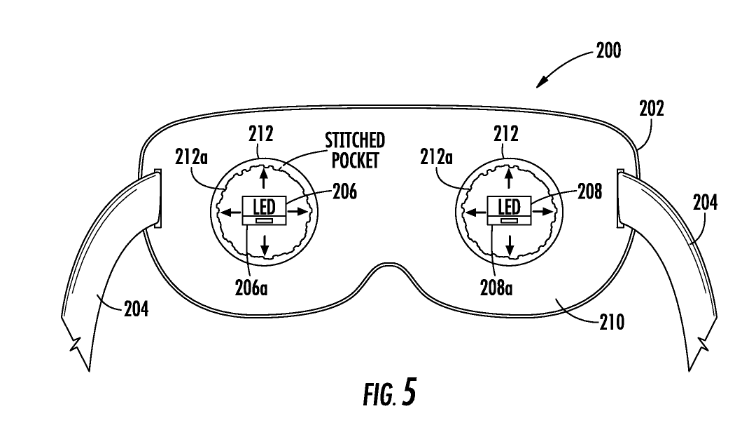

FIG. 5 is an example of a sleep mask 200 that can be used in accordance with a non-limiting example. The sleep mask 200 includes a flexible eye cover 202 that can be made from a variety of different flexible materials, including different fabrics and materials, but generally will be a material that is lightweight and comfortable to the user while blocking out the light. It may include an eye cavity or it can be formed without an eye cavity and, in an example, it is shaped as a more conventional sleep mask as illustrated. It can be made from different types of fabric, including polyester, cotton or other natural or man-made materials. The sleep mask 200 is formed to cover the eyes and in another example may be formed from a soft, moldable material that could also fit the contours of the face. An elastic band 204 or other type of band can be used to hold the sleep mask on the face.

The flexible eye cover 202 is configured to be worn by a pregnant female and configured to cover and shield the eyes of the wearer from ambient light. A source of light is carried by the flexible eye cover 202 and positioned to emit light onto the eyelids of the wearer when asleep and penetrate the eyelids when closed. In a preferred example, the source of light is between about 450 to 500 nm and the light irradiance is between about 80 to 140 W/m². The flexible eye cover as noted before can be formed from fabric and the source of light as illustrated is formed as two light emitting diodes (LED's) 206, 208 carried by the flexible eye cover and positioned at a location where a LED can emit light onto the eyelids of the wearer when asleep.

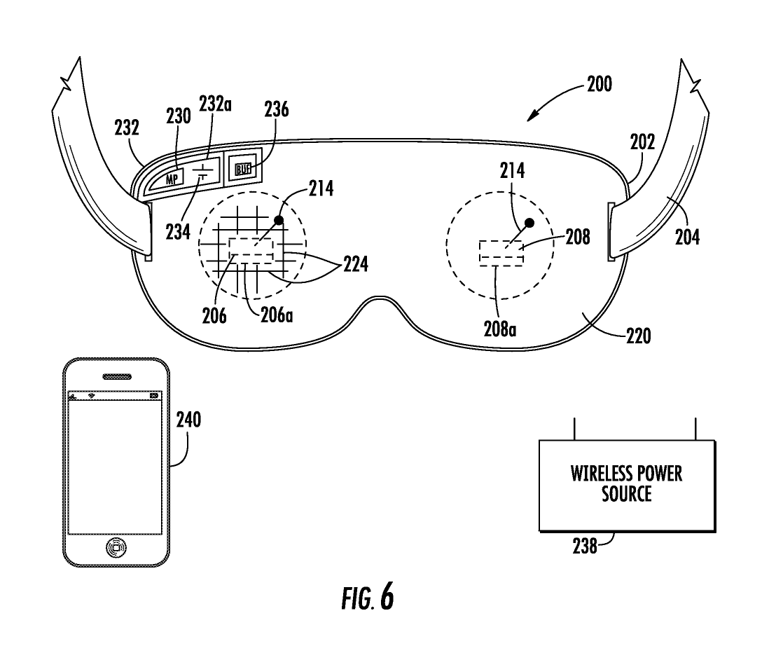

As illustrated in FIG. 5, the rear surface 210 of the flexible eye cover 202 engages the face of the wearer. A pocket 212 is formed on the rear surface at each location in an area generally where the flexible eye cover engages the eyes. The pocket 212 in this example is a circular piece of cloth stitched into the area generally where the eye cover engages the eyes, but could be formed using many different techniques. A LED 206, 208 is positioned within each pocket 212 and is moveable within the pocket to allow it to be positioned to emit light onto the eyelids of the wearer when asleep. In this example as illustrated, the pocket is shown as partially cut away 212a to show the inside of the pocket and the LED 206, 208 positioned within the respective pocket. For example, the LED can be moved within the pocket 212 and positioned by means of an adjuster member 214 as shown in the front view of FIG. 6 and shows a short rod member as the adjuster member extending outward from the front face 220 of the flexible eye cover at the pocket and configured to be grabbed by the wearer to move or manipulate manually a respective LED within the respective pocket into a position adjacent an eye to ensure that the light from the LED is emitted onto the closed eyelid of the wearer when asleep.

The front surface of the cloth or other material forming the pocket 212 may include guide tracks such as fine cuts 224 to allow the adjuster member 214 to be moved vertically or horizontally and position the LED in a desired location that is amenable for use by the user or pregnant female to allow the best position where light shines onto the eyelid. The pocket 212 can be positioned on the front or rear surface of the flexible eye cover and in the illustrated example is shown on the rear surface, but can be on the front surface. The pocket cloth can be made from a different material than that used for the flexible eye cover and can be made more clear to allow the LED light to pass through the material more readily.

A processor 230 or other controller is connected to each LED 206, 208 by wired or wireless connection and configured to activate the LED 206, 208 in a manner to decrease endogenous melatonin levels and aid in regulating uterine contractions in the pregnant female. The processor 230 may be carried in another pocket 232 formed in this example as shown in FIG. 6 in a similar manner as pockets 212. It is shown in a partial cut away 232a and may include a battery 234 or a wireless power inductor or receiver 236 connected to the processor 230 that receives wireless power through a separate wireless power source 238. The LED's 206, 208 may include a small wireless power module 206a, 208a that receives wireless power such that the wireless power itself drives the LED in a predetermined manner with a ramp up of power and intensity and pulsing. A wired connection may be used, of course. Programming of the processor may be through another controller such as a wireless device 240 as illustrated such as a cellular phone, or be preprogrammed when the sleep mask is purchased to be used by the user or pre-programmed by a medical specialist especially for the user as a pregnant female.

FIG. 6 shows an example of the wireless device 240 that can be used to program the processor with a special application program that could be downloaded on an i-phone as an example. A wireless power source 238 is illustrated as shown in FIG. 6. In an example, the processor is programmed so that the illumination is increased over a time period of between about 5 to 30 seconds. The light may be pulsed in discrete on and off cycles such as between 1 to 3 seconds per minute for about 30 to about 60 minutes as noted before.

The processor 230 as a controller may emit pulses at certain times of the night for a certain length of time. The processor 230 can be user programmable via the separate wireless device 240 or preprogrammed when purchased by a user. A mobile device such as a cellular phone, I-phone or other wireless devices is used as a controller in one example to transmit information to the processor for alternate timings and light intensity profiles. Any battery 234 as used would be small for LED actuation and could be rechargeable through a recharge port in an example. White light would be difficult since it may not be feasible especially with a partner sleeping near the user and it could be bothersome to the user. The blue light is a better light and just as effective as white light and has been found effective to suppress melatonin. Also, white light may not penetrate the eyelids as well as the blue light does. The cycling can range from zero power up to full power in ten seconds as an example and can remain on for a minute or half a minute and then go off again through the night. It is possible to operate the mask with a phase response curve to shift the clock eastward or westward by a predetermined number of hours depending on when the light is given. If the light is given in the early evening hours, then time is truncated opposite from light given in the early morning to truncate for the natural low point. It is possible to accelerate the clock. In one example, light pulses are given in the evening and morning for one-half hour to one hour each time and with intermittent ramping and intermittent operation to reduce the truncation. These times can vary.

There are different types of wireless power systems that could be implemented in accordance with a non-limiting example. It is possible to use inductive coupling that uses magnetic fields that are a natural part of the current movement through a wire. For example, when the electrical current moves through a wire, it creates a circular magnetic field around the wire and bends the wire into a coil that amplifies the magnetic field. The more loops the coil has, the larger the field will be produced. When a second coil of wire is placed in the magnetic field, the field can include a current in the wire. It is also possible to use resonance and wireless power conduction can take place differently when the electromagnetic fields around the coils resonate at the same frequency. The inductor can be formed as a curved coil of wire and a capacitance plate can hold a charge and attach to each end of the coil. When electricity travels through the coil, it resonates and the resonant frequency is a product of the inductance of the coil and capacitance of the plates. The electricity can tunnel from one coil to the other as it travels along the electromagnetic wave if both have the same resonant frequency. Electromagnetic induction is proportional to the intensity of the current and voltage in the conductor, which produces the fields and to the frequency. Other wireless power techniques may be used.

The invention has been described above with reference to preferred embodiments. Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described. However, the skilled should understand that the methods and materials used and described are examples and may not be the only ones suitable for use in the invention. Various modifications of the embodiments described here can be made without departing from the spirit and scope of the invention as described above and as defined in the appended claims.

### REFERENCES

1. Plaut S M, Grota L J, Ader R, Graham C W. Effects of handling and the light-dark cycle on time of parturition in the rat. Lab Anim Care. 1970; 20(3):447-453.
2. Boer K, Lincoln D W, Swaab D F. Effects of electrical stimulation of the neurohypophysis on labour in the rat. J Endocrinol. 1975; 65(2):163-176.
3. Lincoln D W, Porter D G. Timing of the photoperiod and the hour of birth in rats. Nature. 1976; 260(5554):780-781.
4. Siegel H I, Greenwald G S. Prepartum onset of maternal behavior in hamsters and the effects of estrogen and progesterone. Horm Behav. 1975; 6(3):237-245.
5. Glattre E, Bjerkedal T. The 24-hour rhythmicity of birth: a population study. Acta Obstet Gynecol Scand. 1983; 62:31-36.
6. Cooperstock M, England J E, Wolfe R A. Circadian incidence of labor onset hour in preterm birth and chorioamnionitis. Obstet Gynecol. 1987; 70(6):852-855.
7. Cagnacei A, Soldani R, Melis G B, Volpe A. Diurnal rhythms of labor and delivery in women: modulation by parity and seasons. Am J Obstet Gynecol. 1998; 178(1 pt 1):140-145.
8. Lindow S W, Jha R R, Thompson J W. 24-hour rhythm to the onset of preterm labour. Br J Obstet Gynecol. 2000; 107(9):1145-1148.
9. Vatish M, Steer P J, Blanks A M, Hon M, Thornton S. Diurnal variation is lost in preterm deliveries before 28 weeks of gestation. Br J Obstet Gynecol. 2010; 117(6):765-767.
10. Jams J D, Newman R B, Thom E A, et al. Frequency of uterine contractions and the risk of spontaneous preterm delivery. N Engl J Med. 2002; 346(4):250-255.
11. Harbert G M Jr. Biorhythms of the pregnant uterus (Macaca mulatta). Am J Obstet Gynecol. 1977; 129(4):401-408.
12. Morgan M A, Silavin S L, Wentworth R A, et al. Different patterns of myometrial activity and 24-h rhythms in myometrial contractility in the gravid baboon during the second half of pregnancy. Biol Reprod. 1992; 46(6):1158-1164.
13. Honnebier M B O M, Myers T, Figueroa J P, Nathanielsz P W. Variations inmyometrial response to intravenous oxytocin administration at different times of the day in the pregnant rhesus monkey. Endocrinology. 1989; 125(3):1498-1503.
14. Ducsay C A, Yellon S M. Photoperiod regulation of uterine activity and melatonin rhythms in the pregnant rhesus macaque. Biol Reprod. 1991; 44(6):967-974.
15. Main D M, Grisso J A, Wold T, Snyder E S, Holmes J, Chiu G. Extended longitudinal study of uterine activity among low-risk women. Am J Obstet Gynecol. 1991; 165(5 pt 1):1317-1322.
16. Zahn V, Hattensperger W. Circadian rhythm of pregnancy contractions. Z Geburtshilfe Perinatol. 1993; 197(1):1-10.
17. Farber D M, Giussani D A, Jenkins S L, et al. Timing of the switch from myometrial contractures to contractions in late-gestation pregnant rhesus monkeys as recorded by myometrial electromyograrn during spontaneous term and androstenedione-induced labor. Biol Reprod. 1997; 56(2):557-562.
18. Prasai M J, Pemicova I, Grant P J, Scott E M. An endocrinologist's guide to the clock. J Clin Endocrinol Metab. 2011; 96(4):913-922.
19. Messager S, Hazlerigg D G, Mercer J G, Morgan P J. Photoperiod differentially regulates the expression of Per1 and ICER in the pars tuberalis and the suprachiasmatic nucleus of the Siberian hamster. Eur J Neurosci. 2000; 12(8):2865-2870.
20. von Gall C, Garabette M L, Kell C A, et al. Rhythmic gene expression in pituitary depends on heterologous sensitization by the neurohormone melatonin. Nat Neurosci. 2002; 5(3):234-238.
21. Imbesi M, Dirim D A, Yildiz S, et al. The melatonin receptor MT1 is required for the differential regulatory actions of melatonin on neuronal clock gene expression in striatal neurons in vitro. J Pineal Res. 2009; 46(1):87-94.
22. Balsalobre A. Clock genes in mammalian peripheral tissues. Cell Tissue Res. 2002; 309(1):193-199.
23. Dickmeis T. Glucocorticoids and the circadian clock. J Endocrinol. 2009; 200(1):3-22.
24. Kiessling S, Eichele G, Oster H. Adrenal glucocorticoids have a key role in circadian resynchronization in a mouse model of jet lag. J Clin Invest. 2010; 120(7):2600-2609.
25. Figueiro, M. G., Bierman, A., and Rea, M. S. A train of blue light pulses delivered through closed eyelids suppresses melatonin and phase shifts the human circadian system. Nature and Science of Sleep 2013:5 133-41.

1-24. (canceled)

25. A sleep mask, comprising:
a flexible eye cover configured to be worn by a late term pregnant female and configured to cover and shield the eyes of the wearer from ambient light;
a source of light having a wavelength of about 450 to 500 nm and carried by the flexible eye cover and positioned to emit light having a light irradiance between about 80 to 140 W/m² onto the eyelids of the wearer when asleep and penetrate the eyelids when closed; and
a processor connected to the source of light and configured to activate the light in a manner to decrease uterine contractions at night in the late term pregnant female and suppress the regular nocturnal endogenous melatonin secretions of the late term pregnant female experiencing uterine contractions.

26. The sleep mask according to claim 25, comprising a battery carried by the flexible eye cover and connected to and supplying power to the processor and source of light.

27. The sleep mask according to claim 25, comprising a wireless power source and said processor and source of light receive wireless power transmitted from the wireless power source.

28. The sleep mask according to claim 25, wherein the illumination of the light is increased over a predetermined period of time.

29. The sleep mask according to claim 28, wherein the illumination is increased over time period of between about 5 to 30 seconds.

30. The sleep mask according to claim 25, wherein the light is pulsed in discrete on and off cycles.

31. The sleep mask according to claim 30, wherein the light is pulsed for about 1 to 3 seconds per minute for about 30 to about 60 minutes.

32. The sleep mask according to claim 25, wherein the flexible eye cover is formed from fabric.

33. The sleep mask according to claim 25, wherein the source of light comprises a light emitting diode (LED) carried by the flexible eye cover and positioned at a location where an LED can emit light onto the eyelids of the wearer when asleep.

34. A sleep mask, comprising:
a flexible eye cover configured to be worn by a late term pregnant female and having a rear surface that engages the face of the wearer and configured to cover and shield the eyes of the wearer from ambient light when worn by a wearer and a front surface opposite the rear surface, and pocket formed on the rear surface at each location in an area generally where the eye cover engages the eyes;
a light emitting diode (LED) that emits light having a wavelength between about 450 to 500 nm and carried by the flexible eye cover within each pocket and movable within the pocket to be positioned to emit light having a light irradiance between about 80 to 140 W/m² onto the eyelids of the wearer when asleep; and
a processor connected to each LED and configured to activate the LED in a manner to decrease uterine contractions at night in the late term pregnant female, and suppressing the regular nocturnal endogenous melatonin secretions of the late term pregnant female experiencing uterine contractions.

35. The sleep mask according to claim 34, comprising a LED position adjustor member connected to each LED and extends outward from the front face of the flexible eye cover and configured to be engaged manually by the wearer and manipulate a respective LED into a position adjacent an eye to insure that the light from the LED is emitted onto the closed eyelid of the wearer when asleep.

36. The sleep mask according to claim 34, comprising a battery carried by the flexible eye cover and connected to and supplying power to the processor and source of light.

37. The sleep mask according to claim 34, comprising a wireless power source and said processor and source of light receive wireless power transmitted from the wireless power source.

38. The sleep mask according to claim 34, wherein the illumination of the light is increased over a predetermined period of time.

39. The sleep mask according to claim 38, wherein the illumination is increased over time period of between about 5 to 30 seconds.

40. The sleep mask according to claim 34, wherein the light is pulsed in discrete on and off cycles.

41. The sleep mask according to claim 40, wherein the light is pulsed for between 1 to 3 seconds per minute for about 30 to about 60 minutes.

42. The sleep mask according to claim 34, wherein the flexible eye cover is formed from fabric.

43. A method of regulating uterine contractions in a late term pregnant female, comprising:
providing a flexible eye cover that is worn by the late term pregnant female and covers and shields the eyes of the late term pregnant female from ambient light;
emitting light having a wavelength of about 450 to 500 nm and a light irradiance between about 80 to 140 W/m² from a light source carried by the flexible eye cover onto the eyelids of the late term pregnant female when asleep to penetrate the eyelids when closed; and
activating the light from a processor carried by the flexible eye cover and connected to the light source in a manner to decrease uterine contractions at night in the late term pregnant female, and suppressing the regular nocturnal endogenous melatonin secretions of the late term pregnant female experiencing uterine contractions.

44. The method according to claim 43, comprising forming the light source as a light emitting diode (LED).

45. The method according to claim 44, comprising mounting a LED on the flexible eye cover at each location in an area generally where the flexible eye cover engages a respective left and right eye when the flexible eye cover is worn by a wearer, and adjusting the position of each LED such that light from a respective LED will be emitted into the respective left and right eyelids of the wearer when asleep.

46. The method according to claim 43, comprising increasing the illumination of the light over a time period between about 5 to about 30 seconds.

47. The method according to claim 43, comprising pulsing the light in discrete on and off cycles.

48. The method according to claim 47, comprising pulsing the light for about 1 to about 3 seconds per minute for about 30 to about 60 minutes.

49. The method according to claim 43, comprising powering the light source and processor from a battery carried by the flexible eye cover and connected to the processor and light source.

50. The method according to claim 43, comprising powering the light source and processor from a wireless power source external to the flexible eye cover, and transmitting a wireless power signal from the wireless power source to the light source and processor that each receive the wireless power signal therefrom.
